(19) United States
(12) Patent Application Publication
(10) Pub. No.: US 2004/0038985 A1
(43) Pub. Date: Feb. 26, 2004

(54) Crystal forms of 1- [6-chloro-5-(trifluoromethly) -2-pyridinyl] piperazine hydrochloride

(76) Inventors: Patrick Carlier, Saint Andre Le Cog (FR); Petrus Josef Cornelis Maria Hoof Van, Postbus 20 (NL); Josef Cornelius Johannes Muyrers, Postbus 20 (NL)

Correspondence Address: William M Blackstone, Akzo Nobel Patent Department Intervet Inc, 405 State Street, Millsboro, DE, 19966, US

(21) Appl. No.: 10/398,991

(22) PCT Filed: Oct. 9, 2001
(86) PCT No.: PCT/EP01/11714

(30) Foreign Application Priority Data
Oct. 13, 2000 (NL) 00203528.5

(51) Int. Cl. A61K 31/496; C 07D 4 3/02

(52) U.S. Cl. 514/253.01; 544/360

(57) ABSTRACT

The invention relates to crystal forms A and B of 1-[6-chloro-5-(trifluoromethyl)-2-pyridinyl]piperazine hydrochloride (Org 12962), to methods for the preparation of those crystal forms and to pharmaceutical compositions comprising crystal form B.

The invention relates to new crystal forms A and B of 1-[6-chloro-5-(trifluoromethyl)-2-pyridinyl]piperazine.hydrochloride, to methods for the preparation of those forms and to pharmaceutical compositions comprising crystal form B.

1-[6-Chloro-5-(trifluoromethyl)-2-pyridinyl]piperazine.hydrochloride, which will be referred to as Org 12962, is known from European Patent 370 560 (Akzo Nobel N. V.), and is described as useful in the treatment of disorders of the central nervous system, especially depression (Leysen, D. C. M. I Drugs, 2, 109-120, 1999) and anxiety (Leysen, D. and Kelder, J. Trends in Drug Research II, 49-61, 1998 Elsevier Science B. V., Ed. H. van der Groot). Additionally the compound is potentially useful in the treatment of urinary incontinence (WO 9833504: Akzo Nobel N. V.). Org 12962 is described in EP 370 560 (Table I, compound no 3) as a compound lacking a well defined melting point. There is no teaching on the physical form of the compound.

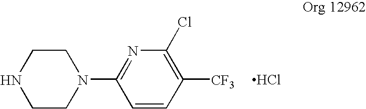

It has now been found that the compound as prepared following the method described in EP 370 560 is polymorphous and consists of a mixture of two crystalline pure forms.

It is generally desirable to prepare therapeutic agents of uniform and defined composition. With regard to polymorphous compounds it may be expected that their biological activity is comparable or identical to that of the crystalline pure forms of which the polymorphous compound consist. Nevertheless, if the polymorphous compound is used as a medicament great drawbacks are associated therewith as compared with its crystalline pure components. The difference in crystal structure can lead to difference in physicochemical parameters such as stability, rate of dissolution, bioavailability, analytical data and the like, which frequently are strongly influenced by the crystal forms in the polymorphous compound. This is all the more important since in practice it is virtually impossible to make each batch of a polymorphous compound exactly identical in respect of its composition. As a consequence of these differences it is often regarded as undesirable to incorporate polymorphous compounds in medicaments and it is-sometimes demanded that only one of the crystalline pure components of the polymorphous compound is used.

It is the aim of the present invention to provide substantially pure crystal forms of the compound Org 12962, which are completely or virtually completely free from the other crystalline forms.

The term “crystalline pure form which is completely or virtually completely free from the other crystalline forms” means a crystal form which contains less than 10% and preferably less than 5% of another crystalline form. It is one aspect of the invention that by using specific crystallization methods three crystalline pure forms, which will be denoted as form A, form B and form C, can be obtained from the polymorphous compound Org 12962.

It has further been found that crystal form B of Org 12962 is the thermodynamically most stable form. The crystal form B is moreover more stable than form A when stored in the dark in mixtures with various pharmaceutical auxilliaries, especially in admixtures comprising lactose and/or cornstarch. In another aspect therefore the present invention relates to the provision of pharmaceutical preparations of solid Org 12962, comprising Org 12962 in the crystalline pure form B. A pharmaceutical composition of this type has the advantage that the reproducibility is appreciably increased and that the physical data, within acceptable limits, are always identical.

When Org 12962 is prepared by treating a solution of the free base of 1-[6-chloro-5-(trifluoromethyl)-2-pyridinyl]piperazine in ethanol with hydrochloric acid, following the general procedure described in EP 370 560, either an amorphous or a polymorphous product, wherein the ratio of amounts of form A and form B will vary widely from batch to batch, is obtained.

Pure crystalline forms A and B can be prepared by crystallizing the hydrochloride salt of 1-[6-chloro-5-(trifluoromethyl)-2-pyridinyl]piperazine under controlled conditions from ethanol or ethanol-water mixtures.,

Pure form A can be prepared by rapidly cooling a concentrated solution of the hydrochloride salt in an ethanol/water mixture from reflux temperature to below 0° C. and initiate nucleation at subzero temperatures (cooling crystallization procedure). Rapid cooling can be effected for instance by keeping the crystallisation flask in ice-acetone (about −10° C.).

Pure form B can be prepared by treating the free base 1-[6-chloro-5-(trifluoromethyl)-2-pyridinyl]piperazine in ethanol (or ethanol-water) solution at reflux temperature with an excess (up to 5 equivalents) of hydrochloric acid whereby the nucleation is initiated at the reflux temperature followed by slowly cooling of the crystallizing salt to ambient temperature (reaction crystallization).

The thermodynamically most stable form B can also be prepared by stirring a suspension of a polymorphous batch of Org 12962 in an ethanol/water mixture until the conversion to form B is complete. This process will take several days when carried out at room temperature [for example: 96 hours for complete conversion of a 20 gram batch in ethanol-water=3:1 at 20° C.]. In a preferred embodiment the suspension is stirred at reflux temperature so that the conversion to form B is complete in several hours [for example: 3 hours for complete conversion of a 1 gram batch in ethanol-water=3:1 at reflux temperature].

A third crystalline form-of Org 12962 can be obtained by crystallization of the compound from 2-methyl-butan-2-ol. This form C is a metastable crystal form, which spontaneously converts to form B, even at −20° C.

The melting points of crystal forms A and B do not differ appreciably. Both pure forms melt between 282-284° C.

The crystal forms of the present invention can be characterized, and thus distinguished from each other, by their X-ray powder diffraction patterns, as well as by their RAMAN spectra.

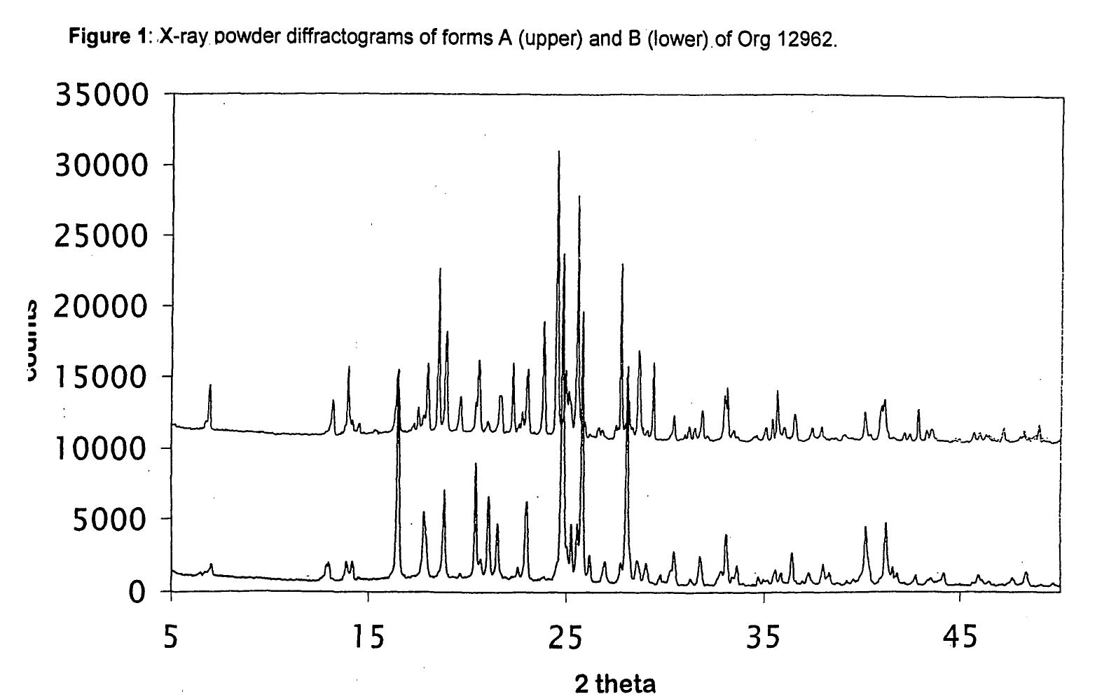

FIG. 1 depicts XRPD spectra for crystal forms A and B of Org 12962. Each of the spectra is characterized by intensity peaks at certain specific values of the diffraction angle 2 theta (θ). Form A has charactistic peaks at 2 theta=17.50°, 17.80°, 23.85°, 24.50°, 25.55°, 27.75° and 29.40°.

Form B is characterized by peaks at 2 theta=20.40°, 21.05°, 24.80°, 25.80° and 28.10°.

From X-ray analysis of a single crystal of form B it was established that the space group of the crystals is P2₁/c, monoclinic, with cell dimensions of a=12.848 Å, b=7.151 Å, c=14.164 Å, β=104.85°, and V=1257.9 Å³.

Similar analysis of form A revealed the crystals to be orthorhombic with space group Pca2₁ and cell dimensions: a=13.823 Å, b=7.226 Å, c=25.256 Å and V=2522.70 Å³.

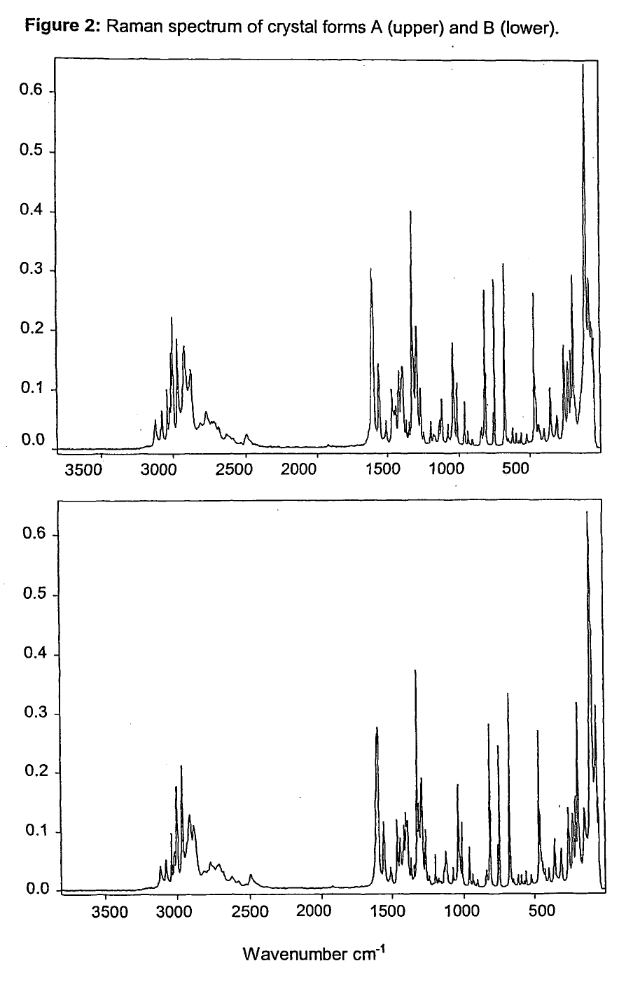

FT Raman spectra of crystal forms A and B are shown in FIG. 2. Each crystal form has characteristic absorption peaks, denoted in Table I. These peaks can be used for the quantitative determination of the amount of crystal form A in pure form B, and the other way around.

TABLE I
Characteristic absorption peaks in Raman spectra
of crystal forms of Org 12962

| Form A | Form B | Form C |
|---|---|---|
| (cm⁻¹) | (cm⁻¹) | (cm₋₁) |
| 82.2 | 104.6 | 187.1 |
| 106.6 | 147.7 | 209.3 |
| 194.7 | 192.8 | 247.3 |
| 211.2 | 209.5 | 345.4 |
| 356.5 | 360.6 | 459.3 |
| 1037.6 | 1035.9 | 680.1 |
| 1268.2 | 1265.3 | 1058.8 |
| 2997.0 | 2997.1 | 1151.1 |
| 3006.5 | 3001.8 | 2853.4 |
| 3122.4 | 3117.2 | 2998.1 |
| | | 3105.4 |

The pharmaceutical preparations of solid Org 12962 according to the invention comprise Org 12962 in the crystalline pure form B in association with one or more pharmaceutically acceptable additives or excipients.

Such pharmaceutical preparations generally take the form of a dosage unit such as a tablet, a capsule or a suppository, but other solid or dry pharmaceutical preparations are included. A preferred pharmaceutical preparation is in the form of a tablet. A tablet may contain in addition to the active principle Org 12962 in the pure crystalline form B, certain excipients, such as diluents, binders, glidants and lubricants, which serve to impart satisfactory processing and compression characteristics to the tablet, as well as disintegrants and flavoring agents, which gives additional desirable physical characteristics to the finished tablet.

Methods for making such dosage units are well known, for example in accordance with standard techniques such as those described in the standard reference, Gennaro et al., Remington's Pharmaceutical Sciences, (18th ed., Mack Publishing Company, 1990, especially Part 8: Pharmaceutical Preparations and Their Manufacture).

A dosage unit of Org 12962, suitable for the treatment of depression, anxiety, obesity, or urinary incontinence, may contain from about 5 to 500 mg of the active ingredient, more usually from about 10-100 mg. A preferred dosage unit may contain 20-40 mg of Orgi 12962 in the crystalline form B, which is to be taken twice a day.

The invention is illustrated by the following examples.

### EXAMPLES

General Procedures:

X-ray powder diffraction (XRPD) spectra were obtained on a Siemens D5000 transmission diffractometer with primary germanium monochromator, Cu-Kα1 radiation, settings 35 kV and 40 mA. The slits used: anti-scatter slit 2 mm, detector slit 0.2 mm. Measuring conditions: step size 0.02°, time per step 10 seconds. The samples were measured in between Scotch tape and were rotated during the measurments with a speed of 15 rpm. The XRPD spectra of crystalline pure forms A and B are depicted in FIG. 1.

FT-Raman spectra were recorded using a Bruker RFS 100⁻ Raman Spectrometer which was equiped with a 1064 nm Nd-YAG laser (Adlas model DPY 421N). Spectra were measured with a resolution of 2 cm⁻¹ using a laser power of 200 mW. Typically, 256 interferograms were collected for each spectrum. The laser spot had a diameter of approximately 30μ at the sample position. Raman spectra of crystal forms A and B are depicted in FIG. 2.

### Example 1

### Preparation of Org 12962.

A: Preparation of 1-[6-chloro-5-(trifluoromethyl)-2-pyridinyl]piperazine

Piperazine (10.337 g; 120 moles) was dissolved in 95% aqeous ethanol (36 l). The solution was heated at reflux, after which a solution of 2,6-dichloro-3-(trifluoromethyl)-pyridine (8640 g; 40 moles) in 95% aqueous ethanol (9 l) was added over 2 hours. The reflux was maintained for another 2 hours. The mixture was cooled to ambient and the precipitated piperazine hydrochloride salt was removed by filtration. The ethanol was removed under vacuum. The residue was dissolved in ethyl acetate (30 l). This solution was washed twice with water (15 l), dried over magnesium sulphate, after which the solvent was removed in vacuo. The residue was poured into water (15 l) while stirring. The resulting solid was filtered off and extensively washed with water.

B: 1-[6-chloro-5-(trifluoromethyl)-2-pvridinyl]piperazine, Hydrochloride Salt [Org 12962]

The wet solid described under A was dissolved in 96% aqueous ethanol (12 l). The solution was once filtered to remove some insoluble material. The filtrate was diluted with 96% aqueous ethanol (15 l), after which hydrogen chloride gas was passed through the solution. During the salification the temperature rose to reflux. After cooling to 0° C., the precipitate is filtered off to give the crude product (7600 g), which was redissolved while heating in 96% aqueous ethanol (11 l) and water (2.7 l). After cooling at ambient temperature, the hydrochloride salt was filtered off, washed with cold ethanol (4 l), and dried at 110° C. in vacuo to give Org 12962 (5500 g; 45.5%) Raman spectroscopy revealed this product to consist of a mixture of crystal forms A (77%) and B (23%).

Elemental analysis:

| | |
|---|---|
| calculated (%) for C₁₀H₁₂N₃Cl₂F₃: | C: 39,75; H: 4.,01; N: 13,91 |
| found (%): | C: 39,81; H: 4.,07; N: 13,85 |

¹H-Nmr (300 MHz in DMSOd6): δ in ppm: 3.26 (4H; triplet J=5.3 Hz); 3.99 (4H; triplet J=5.3 Hz); 7.08 (1H; doublet J=9.0 Hz); 8.04 (1H; doublet J=9.0 Hz); 9.76 (2H; broad singlet: N-H,HCl)

### Example 2

### Preparation of Org 12962 in Pure Crystal Form A

A solution of Org 12962 (35 gram) in 96% aqueous ethanol (1 liter) was rapidly cooled in an ice/acetone bath from reflux temperature to −10° C., while stirring with a magnetic stirring bar. Nucleation occurred spontaneously at subzero temperature. After stiring of the mixture for 2 hours at −10° C. the crystalline material was filtered off, washed with a small amount of cold ethanol and dried in vacuo at room temperature.

### Example 3

### Preparation of Org 12962 in Pure Crystal form B

Polymorphous Org 12962 (10 kg), prepared as descibed in Example 1, was dissolved in a mixture of ethanol (80 l) and water (11 l). The solution was heated to reflux temperature, whereupon solvent was distilled off until the volume of the mixture was reduced to approximately 15 l and crystallization started. The resulting mixture was kept at reflux temperature for 5 hours, after which the solution was slowly cooled to 2±2° C. with a cooling ramp of 17° C./hr. The crystalline mass was filtered off, washed with ethanol (4 l) and dried in vacuo at 60° C. for 24 hours.

### Example 4

### Stability of Crystal Forms A and B in Admixture with Xcipients

Aliquots of Org 12962 (5-10 mg) in crystal form A (a batch prepared as described in Example 2 and which was found to contain 92% form A and 8% form B) were weighed into roundbottomed tubes. To each aliquot an excipient mentioned in Table II was added, followed by 10 μl of water, after which the suspensions were thoroughly mixed.

Aliquots of Org 12962 (5-10 mg) in crystal form B (100% form B) were weighed into roundbottomed tubes. To each aliquot an excipient mentioned in Table II was added, followed by 100 μl of water. Glass beads (250-300 mg) were added to each tube, after which the samples were thoroughly mixed for 1 minute. The residual water was evaporated in vacuo at room temperature. The tubes were stored in the dark for 14 days at either 20° C. or at 60° C. The samples were dissolved in 20.0 ml of a 70:30 mixture of 25 mM phosphate pH 2.6 buffer and acetonitrile. Dissolution was effected by keeping the tubes in a sonifier for 20 minutes.

Quantitative analysis of the amount of Org 12962 per tube was done by high performance liquid chromatography (hplc) using either of the following validated methods: (1) using a Symmetry Shield Reverse Phase RP 18 column (150×4.6 mm), which was operated at 40° C., using a flow rate of 1.0 ml/min, and as eluent a 69:31 (v/v) mixture of 25 mM phosphate buffer pH 2.6 buffer, also containing 15 mM octanesulphonic acid, and acetonitrile, or (2) using a Lichrospher 60 RP Select B column (125×4.0 mm), which was operated at ambient temperature, using a flow rate of 1.5 ml/min, and as eluent a 55:45 (v/v) mixture of methanol and water, also containing 5 mM octanesulphonic acid. The content of Org 12962 remaining in the samples after storage is given in Table II as the percentage (%) of the initial amount.

TABLE II
Stability of Org 12962 in admixture with excipients

| Storage conditions | |
|---|---|
| 14 days | 14 days |
| 20° C./dark | 60° C./dark |

| | I | II | I | II |
|---|---|---|---|---|
| EXCIPIENT | % | % | % | % |
| Lactose (80 mg)* | 94.5 | 99.6 | 91.2 | 97.4 |
| HPC (2 mg) | 98.0 | 100.0 | n.d. | 96.5 |
| Cornstarch (10 mg) | 100.0 | 100.1 | 82.6 | 97.9 |
| Mg-stearate (0.5 mg) | 97.9 | 99.9 | 93.8 | 97.9 |
| Primojel ® (4 mg) | 95.0 | 100.0 | 94.6 | 96.0 |
| Talc (0.3 mg) | 99.2 | 99.9 | 94.5 | 100.1 |
| Titanium dioxide (0.2 mg) | 96.0 | 99.8 | 97.5 | 92.9 |
| HPMC (1.3 mg) | 95.0 | 100.0 | 91.8 | 97.8 |
| PEG 400 (0.3 mg) | 96.5 | 99.8 | 92.5 | 96.3 |

I: crystalline Org 12962: 92% form A and 8% form B;
II: crystalline Org 12962: 100% form B;
*: the amount (in mg) of excipient added to 5.0 mg of Org 12962 is quoted in parenthesis
HPC: hydroxypropylcellulose
HPMC: hydroxypropylmethylcellulose
PEG: polyethylene glycol

The data in Table II indicates that Org 12962 in admixture with pharmaceutical excipients is more stable when in the pure crystal form B as compared with pure crystal form A. When storage was at the stress condition of 60° C., the improved stability of the pure crystal form B was especially notable when in admixture with lactose and with cornstarch, 2 major components of pharmaceutical formulations of Org 12962.

### Example 5

### Pharmaceutical Formulations Comprising Pure Crystal Form B of Org 12962

Tablets of Org 12962, having the following compositions were prepared:

| Ingredients | Mg | Mg |
|---|---|---|
| Org 12962 form B | 10.0 | 100.0 |
| Hydroxypropylcellulose | 4.0 | 8.0 |
| (HPC) | | |
| Corn starch | 20.0 | 40.0 |
| Colloidal silicon dioxide | 3.0 | 6.00 |
| Magnesium stearate | 1.0 | 2.0 |
| Lactose 200 M to a total | 200 | 400 |
| tablet weight of | | |

Org 12962 is homogeneously mixed with the filling agent lactose and the disintegrant corn starch, giving a blend which is granulated in a low shear operation with a mucilage of the binder hydroxypropylcellulose. The moist mass is screened, dried in a fluidized bed, screened again and finally admixed with the colloidal silicon dioxide and the lubricant magnesium stearate. The resulting granulate is compressed to tablet cores.

1. The compound 1-[6-chloro-5-(trifluoromethyl)-2-pyridinyl]piperazine.hydrochloride (Org 12962), characterized in that the compound is in a pure crystal form, which is completely or virtually completely free from other crystalline forms.

2. The compound according to claim 1, having the pure crystal form A which is characterized by an X-ray powder diffraction pattern obtained with CuKα1 radiaton with peaks at values of 2-theta (2θ) of 17.50°, 17.80°, 23.85°, 24.50°, 25.55°, 27.75° and 29.40° and by a Raman absorption spectrum with peaks at 82.2 cm⁻¹, 106.6 cm⁻¹, 194.7 cm⁻¹, 211.2 cm⁻¹, 356.5 cm⁻¹, 1037.6 cm⁻¹, 1268.2 cm⁻¹, 2997.0 cm⁻¹, 3006.5 cm⁻¹ and 3122.4 cm⁻¹.

3. The compound according to claim 1, having the pure crystal form B which is characterized by an X-ray powder diffraction pattern obtained with CuKα1 radiaton with peaks at values of 2-theta (2θ) 20.40°, 21.05°, 24.80°, 25.80° and 28.10° and by a Raman absorption spectrum with peaks at 104.6 cm⁻¹, 147.7 cm⁻¹, 192,8 cm⁻¹, 209.5 cm¹, 360,6 cm⁻¹, 1035.9 cm⁻¹, 1265.3 cm⁻¹, 2997.1 cm⁻¹, 3001.8 cm¹ and 3117.2 cm⁻¹.

4. A method of preparation of the compound according to claim 2, characterized in that a concentrated solution of 1-[6-chloro-5-(trifluoromethyl)-2-pyridinyl]piperazine.hydrochloride salt in an ethanol/water mixture is rapidly cooled to below 0° C. after which spontaneous nucleation is initiated.

5. A method of preparation of the compound according to claim 3, characterized in that a concentrated solution of 1-[6-chloro-5-(trifluoromethyl)-2-pyridinyl]piperazine in an ethanol-water solution is treated at reflux temperature with an excess of hydrochloric acid whereby the nucleation is initiated at reflux temperature, after which the solution is slowly cooled to ambient temperature.

6. A method of preparation of the compound according to claim 3, characterized in that a suspension of polymorphous 1-[6-chloro-5-(trifluoromethyl)-2-pyridinyl]piperazine.hydrochloride in an ethanol/water mixture is kept until conversion to form B is complete.

7. The method according to claim 6 wherein the suspension is kept at the reflux temperature.

8. A pharmaceutical composition comprising a pharmaceutically acceptable excipient and Org 12962, characterized in that Org 12962 is in the substantially pure crystal form B having an X-ray powder diffraction pattern obtained with CuKα1 radiaton with peaks at values of 2-theta (2θ) 20.40°, 21.05°, 24.80°, 25.80° and 28.10° and a Raman absorption spectrum with absorption peaks at 104.6 cm⁻¹, 147.7 cm¹ 192,8 cm⁻¹, 209.5 cm¹, 360,6 cm⁻¹, 1035.9 cm⁻¹, 1265.3 cm¹, 2997.1 cm⁻¹, 3001.8 cm⁻¹ and 3117.2 cm⁻¹.

9. A method of treating depression, anxiety, obesity, or urinary incontinence in a mammal comprising administering a therapeutically effective amount of 1-[6-chloro-5-(trifluoromethyl)-2-pyridinyl]piperazine.hydrochloride in the pure crystal form B of claim 3.
